SEQUENCE LISTING (3 sequences)

SEQ ID NO: 1 - DNA, 3406 bp, Sesamum indicum L.
  source 1..3406: mol_type = genomic DNA
gcttctgaattcatcccttcaaagctaaatcctcctctttcaccagctcaagggttgtacttcttctcagagtacattttgatcatggatttgagccatcacaggttcacaaacaccacttgggagccatacaacattcccatggatcaaggccatctgctccaagatcaaaccccttttgatcatcatccacacgcacaatggccttactttcctccaattcacagccaaaacgagccgtcggattcctcgcccacgcaactcctcccgtctccgaatttcgaccaaatgggctctgcagccttcaaagcgtcagaagccggagatcatcaagacgacgagcccgaagaggagttgggagccatgaaagagatgatgttcaagatcgcagcaatgcaacccgtcgacatcgacccggccaccatccgcaagcccaggaggcggaacgtccggatcagcgacgacccgcagagcgtcgcggcccgccaccgccgtgagaggatcagtgagaagatccggatcctccagagactggtccccgggggcactaagatggacactgcttccatgctggatgaagccattcgctatgttaagttcttgaagaggcaaatccgcatactccaggccaaccaccaccagccgccgtgcatagggatcgccactcccaccggcgccgccaccgctgaagaatgggtggcggtaacaaccaaggccaccgccaccgccaccgcggcagcagggacgtcgtcttcgtacttgtttggagggaacaacaataacgatggggcaggtgatgatatatcaatcccatatgttttaatttaaccataagttttatatattggtgattagcatttcaccatataaattagtatatataattaattttgatgatcaaacgttgtattatcataaggtttgtatttaaaaattcaaaaaaatccatgtgatgataagaaaaaggtttgttaggatattgttgactaagggggcatcggaataatcacaaaacggttagcctgagttatttattgtgtgtatatatataatatatgcaagaagggctttggccctcacaaacttcttttattggactatattttccctgcccgaaataaaacagtgatgtaagaaaactttgaatatcatgactatactctctctaacacacacgtaaacctgcgtgtacgtgtgtgtgtttgcaggcttgtggaagaaagtatagcaacctgcagagagcatatatatatgtgtgtgtgtgtgtgtgtgttttatccctaaatattatgtggattgatgatgaatttgctggtgtctgatggaccacaagaggccaagaatgattgtgttctttttgcagtagcagacgccactttcactgcatatacatcatcatcatcatccatcgatcaaaccctactcttgcaagaaacttgacgagattttcctcaacaacattaaataacaccacacacgtttcttcttctccctgcattcagacaacaatgttcaaccttatagcacattgtcctgaaatctgcatcgtacaaatacgacttctatgtatacattttccagtgtatatgtgttgcaattgtgtgcatacacatactcagggatgtggcgtgtaaaaggaatatatacatatatagatagataatgtcattgtgtgtaataaattgttaagtgtgtaggataattaaatgtatttttctcctataatttaggataaaaattaaatgccaaatgctaccaaaatataggattagaatactaccctagcaagtcaaaaacaaaataacaaagaacctaagttatgagaccaaaaatatgtttaacccttcgtatgtataattaatgtatatgaccaactaagtcgtgtttattaaacttgggatcttttcttctctaaattcctttttttttttttcaaattttcttctatatattcatgtgagggggaaataacacttttcgtccattaatttagagccttttcgtttttggttcctttaattacagaattctcactagtggttctgcaactcctaaaaaatagcatttttactcctataacacacttttgagctgctaaattggtatccaatgcctgcatgcatagtatgatgaattgtttgttagatactgagtgggagcacagctttatatgtctaattgtatctttactcagtatttgtgcaataaaagtaatacgtaaaatcacacttataggtgttaatttggcttctaaacttattaatttataagatcttataactacatatattttataaaataatttaattatatttttagtctcataattttgatcattcagcatttttagctagtaactaatcatttcggcggtttctgttttcgtgtaaccaggagagatttgatcggatttttctcgtgtttactaaatttgttttcaggtgggcggaaagtggtatgtgctactcaggtgactgtttataattagggcaaatcatcatattggctaattaatgccattttgatgccagaaagaaaaattagccaataggatgattcgccctagttttcaacagacacatgagtggcagatacatttttcgaccatctcgaaagaaatcaggcaaacaggggaaaattgatcaaatttgttctgaatttgttagagaaaatattacatgacaaaattacaatcttaacaagttagaggattaaattttcaaatgaacataattacgggagcaaaaatacaattaagcctataagatatatatgtatatatataagatgtttcttaagctaagttgctagattttattctaaaaagaaggctatttaagtatttgaataaaataaaattacgaaactaattataatacgttattaataatgataaatttgtgactaattttttaaaaaaaataatatatttttaaactaagttgaaaatttttacttttttctgaagaagcttataattataagctccaaatcttagtttccggatcgtataatctcattttccacaaaatttaccaaacactctgcaacatctgaaaattaagttgtatattactttgccctattacaactaccattaattaacgttttatgttctgataattaacatatatatatatatacacacacacacatatgtgtgtgtgtttttcataatgcgatttgcaggtgtggaagcattcaatatggagtaaacttgaagagcacatgaactgctgtcgtcggggtattctcgagttcttcaatattgtgggattctgacttgacccattgagacaaagtgtgtagtagaaacttcagtgtattgggcttaatgttgtaagaacagaagggcccaaagccgccggaaaagagaagattgtgtcagtagaaaattacgatcaag

SEQ ID NO: 2 - protein, 244 aa, Sesamum indicum L.
MDLSHHRFTNTTWEPYNIPMDQGHLLQDQTPFDHHPHAQWPYFPPIHSQNEPSDSSPTQLLPSPNFDQMGSAAFKASEAGDHQDDEPEEELGAMKEMMFKIAAMQPVDIDPATIRKPRRRNVRISDDPQSVAARHRRERISEKIRILQRLVPGGTKMDTASMLDEAIRYVKFLKRQIRILQANHHQPPCIGIATPTGAATAEEWVAVTTKATATATAAAGTSSSYLFGGNNNNDGAGVEAFNME

SEQ ID NO: 3 - DNA, 3441 bp, Sesamum indicum L.
  source 1..3441: mol_type = genomic DNA
aagcccttaaaagctgcagagaacctaatcaaagattgcttctgaattcatcccttcaaagctaaatcctcctctttcaccagctcaagggttgtacttcttctcagagtacattttgatcatggatttgagccatcacaggttcacaaacaccacttgggagccatacaacattcccatggatcaaggccatctgctccaagatcaaaccccttttgatcatcatccacaagcacaatggccttactttcctccaattcacagccaaaacgagccgtcggattcctcgcccacgcaactcctcccgtctccgaatttcgaccaaatgggctctgcagccttcaaagcgtcagaagccggagatcatcaagacgacgagcccgaagaggagttgggagccatgaaagagatgatgttcaagatcgcagcaatgcaacccgtcgacatcgacccggccaccatccgcaagcccaggaggcggaacgtccggatcagcgacgacccgcagagcgtcgcggcccgccaccgccgtgagaggatcagtgagaagatccggatcctccagagactggtccccgggggcactaagatggacactgcttccatgctggatgaagccattcgctatgttaagttcttgaagaggcaaatccgcatactccaggccaaccaccaccagccgccgtgcatagggatcgccactcccaccggcgccgccaccgctgaagaatgggtggcggtaacaaccaaggccaccgccaccgccaccgcggcagcagggacgtcgtcttcgtacttgtttggagggaacaacaataacgatggggcaggtgatgatatatcaatcccatatgttttaatttaaccataagttttatatattggtgattagcatttcaccatataaattagtatataaattaattttgatgatcaaacgttgtattatcataaggtttgtatttaaaaattcaaaaaaatccatgtgatgataagaaaaaggtttgttaggatattgttgactaagggggcatcggaataatcacaaaacggttagcctgagttatttattgtgtgtatatatataatatatgcaagaagggctttggccctcataaacttcttttattggcctatattttccctgcccgaaataaaacagtgatgtaagaaaactttgaatatcatgactatactctctctaacacacacgtaaacctgcgtgtacgtgtgtgtgtttgcaggcttgtggaagaaagtatagcaacctgcagagagcatatatatatatatgtgtgtgtgtgtgtgtgttttatccctaaatattatgtggattgatgatgaatttgctggtgtctgatggaccacaagaggccaagaatgattgtgttcttttgcagtagcagacgccactttcactgcatatacatcatcatcatccatcgatcaaaccctactcttgcaagaaacttggcgagattttcctcaacaacattaaataacaccacacacgtttcttcttctccctgcattcagacaacaatgttcaaccttatagcacattgtcctgaaatctgcatcgtacaaatacgacttctatgtatacattttccagtgtatatgtgttgcaattgtgtgcatacacatactcagggatgtggcgtgtaaaaggaatatatacatatatatatataatgtcaatgtgtgtaataaattgttaagtgtgtaggataattaaatgtattttttctcctataatttaggataaaaattaaataccaaatactaccaaaatataggattagaatactaccctaaaaagtcaaaacaaaaataacaaataacctaaattatgagaccaaaaatatgtttaacccttcatatgtatgacttgtataattaatgtattttaccagctaagtcgtgtttattaaacttgggatcttttcttctctatattcctttttttttttttcaaattttcttctatatattcatgtgagggggaaataacacttttcgtccagtaatttagagccttttcatttttggttcctttagttacaaaattctcattagtggttctgtaactcctaaaaaatagcatttttactcctataacacacttttgagctgctaaattggtatccaatgcctgcatgcatagtatgatgaattgtttgttagatactgagtgggagcacagctttatatgtctaattgtatctttactcaatatttgtgcaataaaagtaatacgtaaaatcacacttataggtgttaatttggcttctaaacttattaatttataagatcttataactacatatataaaataatttaattatatttttagtctcataattttgatcattcagcatttttagctagtaactaatcatttcggcgttttctgttttcgtgtaaccaagagagatttgatcggatttttctcgtgtttactaaatttgttttcaggtgggcgaaaagtggtatgtgctactcaggtgactgtttataattagggcaaatcatcatattggctaattaatgccattttgatgccagaaagaaaattagccaataggatgattcgtcctagttttcaacagacacatgagtggcagatacatttttcgaccatctcgaaagaaatcaggcaaacaggaaaaaattgatcaaatttgttctgaatttgttagagaaaaaattacatgatcaaattacaatcttaacaagttagaggattaaattttcaaatggacaaaattacgggagtaaaaatacaattaagcctataagatatatatgtatatatataagatgtttcttaagctaagttgctagattttattctaaaaagaaggctatttaagtatttgaataaaataaaattacgaaactaattataatacgttattaataatgataaatttgtgactaattttaaaaaaaattaatatatttttaaactaagttgaaaatttttacttttttctgaagaagcttataattataagctccaaatcttagtttccggatcgtataatctcattttccacaaaatttaccaaacactctgcaacatctgaaaattaagttgtatattactttgccctattacaactaccattaattaacgttttatgttctgataattaacatatatatatatacacacacacacacatatgtatgtgtgtttttcataatgcgatttgcaggtgtggaagcattcaatatggagtaaacttgaagagcacatgaactgctgtcgtcggggtattctcgagttcttcaatattgtgggattctgacttgacccattgagacaaagtgtgtggtagaaacttcagtgtattgggcttaatgttgtaagaacagaagggcccaaagccgccggaaaagagaagattgtgtcagtagaaaattacgatcaag

(19) United States
(12) Patent Application Publication
(10) Pub. No.: US 2025/0101449 A1
(43) Pub. Date: Mar. 27, 2025

(54) SESAME SEED SHATTERING RESISTANCE TRAIT REGULATION GENE SIHEC3 AND APPLICATION THEREOF

(71) Applicant: HENAN SESAME RESEARCH CENTER, HENAN ACADEMY OF AGRICULTURAL SCIENCES, Zhengzhou (CN)

(72) Inventors: HAIYANG ZHANG, Zhengzhou (CN); HONGMEI MIAO, Zhengzhou (CN); MING JU, Zhengzhou (CN); QIN MA, Zhengzhou (CN); HENGCHUN CAO, Zhengzhou (CN); ZHANYOU ZHANG, Zhengzhou (CN); GUITING LI, Zhengzhou (CN); YINGHUI DUAN, Zhengzhou (CN); CONG MU, Zhengzhou (CN); QIUZHEN TIAN, Zhengzhou (CN); HUILI WANG, Zhengzhou (CN); LINGLING QIN, Zhengzhou (CN); YINGYING HUANG, Zhengzhou (CN)

(21) Appl. No.: 18/978,226

(22) Filed: Dec. 12, 2024

Related U.S. Application Data
(63) Continuation of application No. PCT/CN2024/075020, filed on Jan. 31, 2024.

(30) Foreign Application Priority Data
Jul. 10, 2023 (CN) 202310838584.9

(51) Int. Cl. C12N 15/82; C12Q 1/6895

(52) U.S. Cl. CPC C12N 15/8262; C12Q 1/6895

(57) ABSTRACT

A sesame shattering resistance trait regulation gene Sihec3 and its application are provided. This gene is located on the third chromosome of sesame, is a recessive control gene, and fully explains the phenotypic variation of the shattering resistance trait. Genetic analysis of the shattering trait was conducted with F₂ and F_{2:3} populations derived from a hybrid combination between a shattering resistance mutant M7 and normal shattering material. The research indicates that the seed shattering resistance trait in the tested population is a recessive trait controlled by a single gene. Based on the self-constructed sesame genome fine map and efficient gene mapping technology, the key gene Sihec3 and its allele SiHEC3 regulating the sesame shattering trait were cloned and compared. A corresponding gene marker named SSR1 for the sesame shattering resistance trait was dig. This invention provides a technical foundation for breeding new sesame varieties with shattering resistance trait.

### CROSS-REFERENCE TO RELATED APPLICATIONS

This application is a continuation of International Application No. PCT/CN2024/075020, filed on Jan. 31, 2024, which claims priority to Chinese Patent Application No. 202310838584.9, filed on Jul. 10, 2023, both of which are hereby incorporated by reference in their entireties.

### TECHNICAL FIELD

The present disclosure relates to the field of sesame molecular genetic breeding technologies, and in particular, to a sesame seed shattering resistance trait regulation gene Sihec3 and an application thereof.

### SEQUENCE LISTING

The present application contains a sequence list which was filed electronically in XML format and is hereby incorporated by reference in its entirety. Besides, the XML copy is created on Dec. 12, 2024, is named “SESAME SHATTERING TRAIT REGULAT GENE SIHEC3 AND APPLICATION THEREOF-Sequence Listing.xml” and is 10,891 bytes in sizes.

### BACKGROUND

Sesame is one of the oldest oilseed crops in the world and an important specific agricultural product with high quality in China. Sesame seeds are enriched with health-beneficial unsaturated fatty acids, including oleic and linoleic acids, as well as antioxidants such as sesamol and sasamlinol. Thus, sesame is earned the name of “the queen of oilseed crops” (Zhang et al., 2019). Currently, sesame is grown in 75 countries around the world, mainly distributed in the tropical and the subtropical regions of Asia, Africa, and Latin America. China is one of the main producing countries of sesame, with an annual cultivation area of 520,000 hectares and a total of approximately 650,000 tons, accounting for 10-25% of the global output (FAO statistics from 2000-2018). Due to its high yield level and high seed quality, China has the prominent advantages in sesame production, processing, and worldwide trade.

However, at present, sesame cultivation and harvesting processes are mainly performed by manual due to the limits on planting scale, variety characteristics, and the availability of specialized machinery. The low level of mechanization significantly hinders the sustainable growth of the global sesame industry. As to sesame varieties, the key challenge for sesame mechanization cultivation is that most varieties are common with capsule dehiscence and seed shattering traits, which directly causes seeds loss as capsules mature and open. When harvesting is not carried out in time, yield losses can exceed 50%. This character of sesame varieties is incompatible with the requirement for harvest mechanization. Consequently, enhancing the shattering resistance in sesame variety is crucial for improving the mechanization of sesame production.

### SUMMARY

Based on the obtained mutant material with seed shattering resistance phenotype, sesame reference genome map and gene cloning technology, the present application discloses a gene Sihec3 regulating the seed shattering resistance trait in sesame. This gene supplies a technical foundation for breeding new sesame varieties with high seed shattering resistance trait.
the gene Sihec3, identified on the 3rd chromosome of sesame, is a recessive gene controlling the phenotypic variation associated with the shattering resistance trait.

The technical solution of the present application is list as follows.

The gene Sihec3, identified on the 3rd chromosome of sesame, is a recessive control gene with a 100% explanation for the phenotypic variation associated with the seed shattering resistance trait.
this gene exhibits a deletion with 1049 base pairs in the promoter region ranging from −1 bp˜−1012 bp of and from 1-37 bp in the 5′ UTR region, compared to its allele SiHEC3, which leads to the alteration of gene function. That is to say, as the 1049 bp sequence in the promoter region and part of 5′ UTR region is deleted, the phenotype presents seed shattering resistant.
the genomic DNA (gDNA) of the Sihec3 gene is 3406 bp in length, includes two exons. The sequence of the gDNA is presented in SEQ ID No. 1 with the UTR region consisting of two parts: 1-84 bp and 3217-3406 bp, and the sequence is as follows:

GCTTCTGAATTCATCCCTTCAAAGCTAAATCCTCCTCTTTCACCAGCTCAAGG
GTTGTACTTCTTCTCAGAGTACATTTTGATCATGGATTTGAGCCATCACAGGTTC
ACAAACACCACTTGGGAGCCATACAACATTCCCATGGATCAAGGCCATCTGCTCCA
AGATCAAACCCCTTTTGATCATCATCCACACGCACAATGGCCTTACTTTCCTCCAAT
TCACAGCCAAAACGAGCCGTCGGATTCCTCGCCCACGCAACTCCTCCCGTCTCCG
AATTTCGACCAAATGGGCTCTGCAGCCTTCAAAGCGTCAGAAGCCGGAGATCATC
AAGACGACGAGCCCGAAGAGGAGTTGGGAGCCATGAAAGAGATGATGTTCAAGA
TCGCAGCAATGCAACCCGTCGACATCGACCCGGCCACCATCCGCAAGCCCAGGAG
GCGGAACGTCCGGATCAGCGACGACCCGCAGAGCGTCGCGGCCCGCCACCGCCG
TGAGAGGATCAGTGAGAAGATCCGGATCCTCCAGAGACTGGTCCCCGGGGGCACT
AAGATGGACACTGCTTCCATGCTGGATGAAGCCATTCGCTATGTTAAGTTCTTGAA
GAGGCAAATCCGCATACTCCAGGCCAACCACCACCAGCCGCCGTGCATAGGGATC
GCCACTCCCACCGGCGCCGCCACCGCTGAAGAATGGGTGGCGGTAACAACCAAG
GCCACCGCCACCGCCACCGCGGCAGCAGGGACGTCGTCTTCGTACTTGTTTGGAG
GGAACAACAATAACGATGGGGCAGGTGATGATATATCAATCCCATATGTTTTAATTT
AACCATAAGTTTTATATATTGGTGATTAGCATTTCACCATATAAATTAGTATATATAATT
AATTTTGATGATCAAACGTTGTATTATCATAAGGTTTGTATTTAAAAATTCAAAAAA
ATCCATGTGATGATAAGAAAAAGGTTTGTTAGGATATTGTTGACTAAGGGGGCATC
GGAATAATCACAAAACGGTTAGCCTGAGTTATTTATTGTGTGTATATATATAATATATG
CAAGAAGGGCTTTGGCCCTCACAAACTTCTTTTATTGGACTATATTTTCCCTGCCCG
AAATAAAACAGTGATGTAAGAAAACTTTGAATATCATGACTATACTCTCTCTAACAC
ACACGTAAACCTGCGTGTACGTGTGTGTGTTTGCAGGCTTGTGGAAGAAAGTATAG
CAACCTGCAGAGAGCATATATATATGTGTGTGTGTGTGTGTGTGTTTTATCCCTAAAT
ATTATGTGGATTGATGATGAATTTGCTGGTGTCTGATGGACCACAAGAGGCCAAGA
ATGATTGTGTTCTTTTTGCAGTAGCAGACGCCACTTTCACTGCATATACATCATCATC
ATCATCCATCGATCAAACCCTACTCTTGCAAGAAACTTGACGAGATTTTCCTCAAC
AACATTAAATAACACCACACACGTTTCTTCTTCTCCCTGCATTCAGACAACAATGTT
CAACCTTATAGCACATTGTCCTGAAATCTGCATCGTACAAATACGACTTCTATGTATA
CATTTTCCAGTGTATATGTGTTGCAATTGTGTGCATACACATACTCAGGGATGTGGC
GTGTAAAAGGAATATATACATATATAGATAGATAATGTCATTGTGTGTAATAAATTGTT
AAGTGTGTAGGATAATTAAATGTATTTTTCTCCTATAATTTAGGATAAAAATTAAATG
CCAAATGCTACCAAAATATAGGATTAGAATACTACCCTAGCAAGTCAAAAACAAAA
TAACAAAGAACCTAAGTTATGAGACCAAAAATATGTTTAACCCTTCGTATGTATAAT
TAATGTATATGACCAACTAAGTCGTGTTTATTAAACTTGGGATCTTTTCTTCTCTAAA
TTCCTTTTTTTTTTTTTCAAATTTTCTTCTATATATTCATGTGAGGGGGAAATAACAC
TTTTCGTCCATTAATTTAGAGCCTTTTCGTTTTTGGTTCCTTTAATTACAGAATTCTC
ACTAGTGGTTCTGCAACTCCTAAAAAATAGCATTTTTACTCCTATAACACACTTTTG
AGCTGCTAAATTGGTATCCAATGCCTGCATGCATAGTATGATGAATTGTTTGTTAGAT
ACTGAGTGGGAGCACAGCTTTATATGTCTAATTGTATCTTTACTCAGTATTTGTGCA
ATAAAAGTAATACGTAAAATCACACTTATAGGTGTTAATTTGGCTTCTAAACTTATTA
ATTTATAAGATCTTATAACTACATATATTTTATAAAATAATTTAATTATATTTTTAGTCT
CATAATTTTGATCATTCAGCATTTTTAGCTAGTAACTAATCATTTCGGCGGTTTCTGT
TTTCGTGTAACCAGGAGAGATTTGATCGGATTTTTCTCGTGTTTACTAAATTTGTTT
TCAGGTGGGCGGAAAGTGGTATGTGCTACTCAGGTGACTGTTTATAATTAGGGCAA
ATCATCATATTGGCTAATTAATGCCATTTTGATGCCAGAAAGAAAAATTAGCCAATA
GGATGATTCGCCCTAGTTTTCAACAGACACATGAGTGGCAGATACATTTTTCGACC
ATCTCGAAAGAAATCAGGCAAACAGGGGAAAATTGATCAAATTTGTTCTGAATTTG
TTAGAGAAAATATTACATGACAAAATTACAATCTTAACAAGTTAGAGGATTAAATTT
TCAAATGAACATAATTACGGGAGCAAAAATACAATTAAGCCTATAAGATATATATGT
ATATATATAAGATGTTTCTTAAGCTAAGTTGCTAGATTTTATTCTAAAAAGAAGGCTA
TTTAAGTATTTGAATAAAATAAAATTACGAAACTAATTATAATACGTTATTAATAATG
ATAAATTTGTGACTAATTTTTTAAAAAAAATAATATATTTTTAAACTAAGTTGAAAAT
TTTTACTTTTTTCTGAAGAAGCTTATAATTATAAGCTCCAAATCTTAGTTTCCGGATC
GTATAATCTCATTTTCCACAAAATTTACCAAACACTCTGCAACATCTGAAAATTAAG
TTGTATATTACTTTGCCCTATTACAACTACCATTAATTAACGTTTTATGTTCTGATAAT
TAACATATATATATATATACACACACACACATATGTGTGTGTGTTTTTCATAATGCGAT
TTGCAGGTGTGGAAGCATTCAATATGGAGTAAACTTGAAGAGCACATGAACTGCT
GTCGTCGGGGTATTCTCGAGTTCTTCAATATTGTGGGATTCTGACTTGACCCATTGA
GACAAAGTGTGTAGTAGAAACTTCAGTGTATTGGGCTTAATGTTGTAAGAACAGAA
GGGCCCAAAGCCGCCGGAAAAGAGAAGATTGTGTCAGTAGAAAATTACGATCAAG.

A protein encoded by the sesame shattering resistance regulation gene Sihec3 is composed of 244 amino acids, with the corresponding amino acid sequence provided in SEQ ID No. 2:

MDLSHHRFTNTTWEPYNIPMDQGHLLQDQTPFDHHPHAQWPYFPPIHSQN
EPSDSSPTQLLPSPNFDQMGSAAFKASEAGDHQDDEPEEELGAMKEMMFK
IAAMQPVDIDPATIRKPRRRNVRISDDPQSVAARHRRERISEKIRILQRL
VPGGTKMDTASMLDEAIRYVKFLKRQIRILQANHHQPPCIGIATPTGAAT
AEEWVAVTTKATATATAAAGTSSSYLFGGNNNNDGAGVEAFNME;

correspondingly, the cDNA sequence of the sesame seed shattering resistance regulation gene Sihec3, corresponding to the shattering resistance phenotype is 735 bp in length. Compared to the cDNA sequence of its allele SiHEC3, there is mutation at 111th nucleotide position where adenine (A) is substituted by cytosine (C). This point mutation leads to an amino acid change at the 37th position of the encoded protein, converting glutamine (Q) to histidine (H). The full cDNA sequence is presented below:

ATGGATTTGAGCCATCACAGGTTCACAAACACCACTTGGGAGCCATACAA
CATTCCCATGGATCAAGGCCATCTGCTCCAAGATCAAACCCCTTTTGATC
ATCATCCACACGCACAATGGCCTTACTTTCCTCCAATTCACAGCCAAAAC
GAGCCGTCGGATTCCTCGCCCACGCAACTCCTCCCGTCTCCGAATTTCGA
CCAAATGGGCTCTGCAGCCTTCAAAGCGTCAGAAGCCGGAGATCATCAAG
ACGACGAGCCCGAAGAGGAGTTGGGAGCCATGAAAGAGATGATGTTCAAG
ATCGCAGCAATGCAACCCGTCGACATCGACCCGGCCACCATCCGCAAGCC
CAGGAGGCGGAACGTCCGGATCAGCGACGACCCGCAGAGCGTCGCGGCCC
GCCACCGCCGTGAGAGGATCAGTGAGAAGATCCGGATCCTCCAGAGACTG
GTCCCCGGGGGCACTAAGATGGACACTGCTTCCATGCTGGATGAAGCCAT
TCGCTATGTTAAGTTCTTGAAGAGGCAAATCCGCATACTCCAGGCCAACC
ACCACCAGCCGCCGTGCATAGGGATCGCCACTCCCACCGGCGCCGCCACC
GCTGAAGAATGGGTGGCGGTAACAACCAAGGCCACCGCCACCGCCACCGC
GGCAGCAGGGACGTCGTCTTCGTACTTGTTTGGAGGGAACAACAATAAC
GATGGGGCAGGTGTGGAAGCATTCAATATGGAGTAA.

An allele SiHEC3 to gene Sihec3 regulating the sesame seed shattering resistance trait is a complete dominant gene. Sesame varieties containing this allele exhibit the typical seed shattering trait;
the gDNA of the allele SiHEC3 has a sequence length of 3441 bp, and contains two exons. The base sequence is provided in SEQ ID No. 3 as follows (UTR region includes two parts: 1-121 bp and 3264-3441 bp). The complete base sequence is as follows:

AAGCCCTTAAAAGCTGCAGAGAACCTAATCAAAGATTGCTTCTGAATTCATCC
CTTCAAAGCTAAATCCTCCTCTTTCACCAGCTCAAGGGTTGTACTTCTTCTCA
GAGTACATTTTGATCATGGATTTGAGCCATCACAGGTTCACAAACACCACTTGGG
AGCCATACAACATTCCCATGGATCAAGGCCATCTGCTCCAAGATCAAACCCCTTTT
GATCATCATCCACAAGCACAATGGCCTTACTTTCCTCCAATTCACAGCCAAAACGA
GCCGTCGGATTCCTCGCCCACGCAACTCCTCCCGTCTCCGAATTTCGACCAAATGG
GCTCTGCAGCCTTCAAAGCGTCAGAAGCCGGAGATCATCAAGACGACGAGCCCGA
AGAGGAGTTGGGAGCCATGAAAGAGATGATGTTCAAGATCGCAGCAATGCAACCC
GTCGACATCGACCCGGCCACCATCCGCAAGCCCAGGAGGCGGAACGTCCGGATCA
GCGACGACCCGCAGAGCGTCGCGGCCCGCCACCGCCGTGAGAGGATCAGTGAGA
AGATCCGGATCCTCCAGAGACTGGTCCCCGGGGGCACTAAGATGGACACTGCTTC
CATGCTGGATGAAGCCATTCGCTATGTTAAGTTCTTGAAGAGGCAAATCCGCATAC
TCCAGGCCAACCACCACCAGCCGCCGTGCATAGGGATCGCCACTCCCACCGGCGC
CGCCACCGCTGAAGAATGGGTGGCGGTAACAACCAAGGCCACCGCCACCGCCAC
CGCGGCAGCAGGGACGTCGTCTTCGTACTTGTTTGGAGGGAACAACAATAACGAT
GGGGCAGGTGATGATATATCAATCCCATATGTTTTAATTTAACCATAAGTTTTATATAT
TGGTGATTAGCATTTCACCATATAAATTAGTATATAAATTAATTTTGATGATCAAACG
TTGTATTATCATAAGGTTTGTATTTAAAAATTCAAAAAAATCCATGTGATGATAAGA
AAAAGGTTTGTTAGGATATTGTTGACTAAGGGGGCATCGGAATAATCACAAAACGG
TTAGCCTGAGTTATTTATTGTGTGTATATATATAATATATGCAAGAAGGGCTTTGGCC
CTCATAAACTTCTTTTATTGGCCTATATTTTCCCTGCCCGAAATAAAACAGTGATGTA
AGAAAACTTTGAATATCATGACTATACTCTCTCTAACACACACGTAAACCTGCGTGT
ACGTGTGTGTGTTTGCAGGCTTGTGGAAGAAAGTATAGCAACCTGCAGAGAGCAT
ATATATATATATGTGTGTGTGTGTGTGTGTTTTATCCCTAAATATTATGTGGATTGATG
ATGAATTTGCTGGTGTCTGATGGACCACAAGAGGCCAAGAATGATTGTGTTCTTTT
GCAGTAGCAGACGCCACTTTCACTGCATATACATCATCATCATCCATCGATCAAACC
CTACTCTTGCAAGAAACTTGGCGAGATTTTCCTCAACAACATTAAATAACACCACA
CACGTTTCTTCTTCTCCCTGCATTCAGACAACAATGTTCAACCTTATAGCACATTGT
CCTGAAATCTGCATCGTACAAATACGACTTCTATGTATACATTTTCCAGTGTATATGT
GTTGCAATTGTGTGCATACACATACTCAGGGATGTGGCGTGTAAAAGGAATATATAC
ATATATATATATAATGTCAATGTGTGTAATAAATTGTTAAGTGTGTAGGATAATTAAAT
GTATTTTTTCTCCTATAATTTAGGATAAAAATTAAATACCAAATACTACCAAAATATA
GGATTAGAATACTACCCTAAAAAGTCAAAACAAAAATAACAAATAACCTAAATTAT
GAGACCAAAAATATGTTTAACCCTTCATATGTATGACTTGTATAATTAATGTATTTTA
CCAGCTAAGTCGTGTTTATTAAACTTGGGATCTTTTCTTCTCTATATTCCTTTTTTTTT
TTTTCAAATTTTCTTCTATATATTCATGTGAGGGGGAAATAACACTTTTCGTCCAGTA
ATTTAGAGCCTTTTCATTTTTGGTTCCTTTAGTTACAAAATTCTCATTAGTGGTTCTG
TAACTCCTAAAAAATAGCATTTTTACTCCTATAACACACTTTTGAGCTGCTAAATTG
GTATCCAATGCCTGCATGCATAGTATGATGAATTGTTTGTTAGATACTGAGTGGGAG
CACAGCTTTATATGTCTAATTGTATCTTTACTCAATATTTGTGCAATAAAAGTAATAC
GTAAAATCACACTTATAGGTGTTAATTTGGCTTCTAAACTTATTAATTTATAAGATCT
TATAACTACATATATAAAATAATTTAATTATATTTTTAGTCTCATAATTTTGATCATTCA
GCATTTTTAGCTAGTAACTAATCATTTCGGCGTTTTCTGTTTTCGTGTAACCAAGAG
AGATTTGATCGGATTTTTCTCGTGTTTACTAAATTTGTTTTCAGGTGGGCGAAAAGT
GGTATGTGCTACTCAGGTGACTGTTTATAATTAGGGCAAATCATCATATTGGCTAATT
AATGCCATTTTGATGCCAGAAAGAAAATTAGCCAATAGGATGATTCGTCCTAGTTTT
CAACAGACACATGAGTGGCAGATACATTTTTCGACCATCTCGAAAGAAATCAGGC
AAACAGGAAAAAATTGATCAAATTTGTTCTGAATTTGTTAGAGAAAAAATTACATG
ATCAAATTACAATCTTAACAAGTTAGAGGATTAAATTTTCAAATGGACAAAATTACG
GGAGTAAAAATACAATTAAGCCTATAAGATATATATGTATATATATAAGATGTTTCTTA
AGCTAAGTTGCTAGATTTTATTCTAAAAAGAAGGCTATTTAAGTATTTGAATAAAAT
AAAATTACGAAACTAATTATAATACGTTATTAATAATGATAAATTTGTGACTAATTTT
AAAAAAAATTAATATATTTTTAAACTAAGTTGAAAATTTTTACTTTTTTCTGAAGAA
GCTTATAATTATAAGCTCCAAATCTTAGTTTCCGGATCGTATAATCTCATTTTCCACA
AAATTTACCAAACACTCTGCAACATCTGAAAATTAAGTTGTATATTACTTTGCCCTA
TTACAACTACCATTAATTAACGTTTTATGTTCTGATAATTAACATATATATATATACAC
ACACACACACATATGTATGTGTGTTTTTCATAATGCGATTTGCAGGTGTGGAAGCAT
TCAATATGGAGTAAACTTGAAGAGCACATGAACTGCTGTCGTCGGGGTATTCTC
GAGTTCTTCAATATTGTGGGATTCTGACTTGACCCATTGAGACAAAGTGTGTG
GTAGAAACTTCAGTGTATTGGGCTTAATGTTGTAAGAACAGAAGGGCCCAAA
GCCGCCGGAAAAGAGAAGATTGTGTCAGTAGAAAATTACGATCAAG.

The amino acid sequence encoded by the allele SiHEC3 contain 244 amino acids with the amino acid sequence as following:

MDLSHHRFTNTTWEPYNIPMDQGHLLQDQTPFDHHPQAQWPYFPPIHSQN
EPSDSSPTQLLPSPNFDQMGSAAFKASEAGDHQDDEPEEELGAMKEMMFK
IAAMQPVDIDPATIRKPRRRNVRISDDPQSVAARHRRERISEKIRILQRL
VPGGTKMDTASMLDEAIRYVKFLKRQIRILQANHHQPPCIGIATPTGAAT
AEEWVAVTTKATATATAAAGTSSSYLFGGNNNNDGAGVEAFNME;

correspondingly, the cDNA corresponding to the allele SiHEC3 has 735 bp with the base sequence detailed as following:

ATGGATTTGAGCCATCACAGGTTCACAAACACCACTTGGGAGCCATACAA
CATTCCCATGGATCAAGGCCATCTGCTCCAAGATCAAACCCCTTTTGATC
ATCATCCACAAGCACAATGGCCTTACTTTCCTCCAATTCACAGCCAAAAC
GAGCCGTCGGATTCCTCGCCCACGCAACTCCTCCCGTCTCCGAATTTCGA
CCAAATGGGCTCTGCAGCCTTCAAAGCGTCAGAAGCCGGAGATCATCAAG
ACGACGAGCCCGAAGAGGAGTTGGGAGCCATGAAAGAGATGATGTTCAAG
ATCGCAGCAATGCAACCCGTCGACATCGACCCGGCCACCATCCGCAAGCC
CAGGAGGCGGAACGTCCGGATCAGCGACGACCCGCAGAGCGTCGCGGCCC
GCCACCGCCGTGAGAGGATCAGTGAGAAGATCCGGATCCTCCAGAGACTG
GTCCCCGGGGGCACTAAGATGGACACTGCTTCCATGCTGGATGAAGCCAT
TCGCTATGTTAAGTTCTTGAAGAGGCAAATCCGCATACTCCAGGCCAACC
ACCACCAGCCGCCGTGCATAGGGATCGCCACTCCCACCGGCGCCGCCACC
GCTGAAGAATGGGTGGCGGTAACAACCAAGGCCACCGCCACCGCCACCGC
GGCAGCAGGGACGTCGTCTTCGTACTTGTTTGGAGGGAACAACAATAACG
ATGGGGCAGGTGTGGAAGCATTCAATATGGAGTAA.

To amplify the promoter and gene sequences of the seed shattering resistance gene Sihec3 and its allele SiHEC3, and to illustrate the difference between the two sequences, two pairs of PCR primers have been designed for amplification based on the reference genome sequence of sesame variety Yuzhi 11. These primers are as follows:

Primer1F forward primer:
5′-TGACGGTCCGATTTGTAAGGTG-3′,
Primer1R reverse primer:
5′-GAAATTCGGAGACGGGAGG-3′;
Primer2F forward primer:
5′-AGCTAAATCCTCCTCTTTCACCA-3′,
Primer2R reverse primer:
5′-ACAGCGTACAGGTTTCATT-3′;

as PCR amplification is conducted using the two primer pairs, genomic DNAs from germplasm resources with shattering trait or shattering resistance trait are used as templates, respectively.
with the Primer1F/R primer set, the amplification for shattering and shattering resistance materials produces 1392 bp and 2432 bp band, respectively.
with the Primer 2F/R primer set, the amplification for shattering and shattering resistance materials produces 3410 bp and 3408 bp band, respectively.
based on amplicon sequencing and sequence alignment with the reference genome, the 1111 bp upstream sequence and 3406 bp gDNA sequence for the sesame shattering resistance gene Sihec3 and the 2114 bp upstream sequence and 3441 bp gDNA sequence of the allele SiHEC3 are obtained;
sequence comparison results show that the most notable difference between the sesame shattering resistance gene Sihec3 and allele SiHEC3 is that Sihec3 lacks 1012 bp promoter sequence and 37 bp gene sequence, compared to the allele SiHEC3, besides of some bases changes in the promoter region.

It should be explained that, as the primer pairs are designed, the amplification product with the first primer pair Primer1F/R contains the promoter and partial gene sequences, while the amplification product with the second primer pair Primer2F/R contains partial gene and gene downstream sequences; and a specific amplification sequence is as follows.

The PCR amplification with two primer pairs for sesame materials with seed shattering resistance are as follows (4543 bp, including the upstream, UTR, CDS, and intron sequences):

TGACGGTCCGATTTGTAAGGTGATAGGGATGATCGAAAGATTGTTTCCCCAACCT
TGGATGCATTAGACGtTGCTGCTGGAAGTGGCGTGCTACGACCATATTAACGTGATA
CAGGAGGCGTACCACTAGACATAATTAATACAAATTATAGTAAACATTAGTTTTTATA
ATTGCTGAAAAAATTAACCTAATTCAATAATTTATGTTGAATTTATGATTATTGTACT
CATTAATTACTTAAAAATATATAGTTCCTTTGTACACATATACAAACGGGTCAAGCAT
TGCTTTTAACTTCTTTAGCATATCACATAAATTTTTTTGTTATAGTACTACTCCAGTTT
GTCCACTCTTTTTCTGTCAACATTGTCTTCTCTTTACAAATAAGAATGGATAAACCT
GACTAATTCATTCTACTAAGAAAAAATCACTTGATGAGCTAAAAAAGCTAAAGCAA
TGCTTGATCCATTTGTACATGGGCACAAAGAAACTATACATTTTTAATGACTCTTATT
GAAAATATTATTATTGGAACTATTAGCCTAATATTGTGATTATTGACATAATTATGTAA
ATAGTGTTATGTGAATACTAGTCTTGTGATTGTTAATATTATTATGTAAATAATGTTAT
TATTGTGAATATTAGTCTAATTTTGCGATTGTTGACATTATTATGTAAATAATGTTATT
ATTTAGAATATTGAATAACGTTGTGATTATTGATATTATTATGTAAATAATATTATTCTT
GAAAATATTAGCCTAAATTTTGTGATTATTGACATTATTTTGTAAATAATATTATTATT
GAGAATATTGATCTAATTTTATGATTATTGACATTATTAAATAAATAATATTATCATTTG
AAATATTAGCTTAAAGGTTGTGAATACTGACATTGTTATGTAAATAATGTTATTATTG
AAAATTGTAATCGAATTTTGTGATTGTTGACATTATTATGTAAATAATAATATTAATGA
AAAAATGCAAGAATTACATCAATTACACCACTATGTAAATTACACAATATGATACAA
TACACACAAAAATTGGGGTCACGATGTCATATACCAAATTTCATTCCTAATGTGTTC
CAATACTCGCTTCTGAATTCATCCCTTCAAAGCTAAATCCTCCTCTTTCACCAGCTC
AAGGGTTGTACTTCTTCTCAGAGTACATTTTGATCATGGATTTGAGCCATCACAGGT
TCACAAACACCACTTGGGAGCCATACAACATTCCCATGGATCAAGGCCATCTGCTC
CAAGATCAAACCCCTTTTGATCATCATCCACACGCACAATGGCCTTACTTTCCTCCA
ATTCACAGCCAAAACGAGCCGTCGGATTCCTCGCCCACGCAACTCCTCCCGTCTCC
GAATTTCGACCAAATGGGCTCTGCAGCCTTCAAAGCGTCAGAAGCCGGAGATCAT
CAAGACGACGAGCCCGAAGAGGAGTTGGGAGCCATGAAAGAGATGATGTTCAAG
ATCGCAGCAATGCAACCCGTCGACATCGACCCGGCCACCATCCGCAAGCCCAGGA
GGCGGAACGTCCGGATCAGCGACGACCCGCAGAGCGTCGCGGCCCGCCACCGCC
GTGAGAGGATCAGTGAGAAGATCCGGATCCTCCAGAGACTGGTCCCCGGGGGCAC
TAAGATGGACACTGCTTCCATGCTGGATGAAGCCATTCGCTATGTTAAGTTCTTGAA
GAGGCAAATCCGCATACTCCAGGCCAACCACCACCAGCCGCCGTGCATAGGGATC
GCCACTCCCACCGGCGCCGCCACCGCTGAAGAATGGGTGGCGGTAACAACCAAG
GCCACCGCCACCGCCACCGCGGCAGCAGGGACGTCGTCTTCGTACTTGTTTGGAG
GGAACAACAATAACGATGGGGCAGGTGATGATATATCAATCCCATATGTTTTAATTT
AACCATAAGTTTTATATATTGGTGATTAGCATTTCACCATATAAATTAGTATATATAATT
AATTTTGATGATCAAACGTTGTATTATCATAAGGTTTGTATTTAAAAATTCAAAAAA
ATCCATGTGATGATAAGAAAAAGGTTTGTTAGGATATTGTTGACTAAGGGGGCATC
GGAATAATCACAAAACGGTTAGCCTGAGTTATTTATTGTGTGTATATATATAATATATG
CAAGAAGGGCTTTGGCCCTCACAAACTTCTTTTATTGGACTATATTTTCCCTGCCCG
AAATAAAACAGTGATGTAAGAAAACTTTGAATATCATGACTATACTCTCTCTAACAC
ACACGTAAACCTGCGTGTACGTGTGTGTGTTTGCAGGCTTGTGGAAGAAAGTATAG
CAACCTGCAGAGAGCATATATATATGTGTGTGTGTGTGTGTGTGTTTTATCCCTAAAT
ATTATGTGGATTGATGATGAATTTGCTGGTGTCTGATGGACCACAAGAGGCCAAGA
ATGATTGTGTTCTTTTTGCAGTAGCAGACGCCACTTTCACTGCATATACATCATCATC
ATCATCCATCGATCAAACCCTACTCTTGCAAGAAACTTGACGAGATTTTCCTCAAC
AACATTAAATAACACCACACACGTTTCTTCTTCTCCCTGCATTCAGACAACAATGTT
CAACCTTATAGCACATTGTCCTGAAATCTGCATCGTACAAATACGACTTCTATGTATA
CATTTTCCAGTGTATATGTGTTGCAATTGTGTGCATACACATACTCAGGGATGTGGC
GTGTAAAAGGAATATATACATATATAGATAGATAATGTCATTGTGTGTAATAAATTGTT
AAGTGTGTAGGATAATTAAATGTATTTTTCTCCTATAATTTAGGATAAAAATTAAATG
CCAAATGCTACCAAAATATAGGATTAGAATACTACCCTAGCAAGTCAAAAACAAAA
TAACAAAGAACCTAAGTTATGAGACCAAAAATATGTTTAACCCTTCGTATGTATAAT
TAATGTATATGACCAACTAAGTCGTGTTTATTAAACTTGGGATCTTTTCTTCTCTAAA
TTCCTTTTTTTTTTTTTCAAATTTTCTTCTATATATTCATGTGAGGGGGAAATAACAC
TTTTCGTCCATTAATTTAGAGCCTTTTCGTTTTTGGTTCCTTTAATTACAGAATTCTC
ACTAGTGGTTCTGCAACTCCTAAAAAATAGCATTTTTACTCCTATAACACACTTTTG
AGCTGCTAAATTGGTATCCAATGCCTGCATGCATAGTATGATGAATTGTTTGTTAGAT
ACTGAGTGGGAGCACAGCTTTATATGTCTAATTGTATCTTTACTCAGTATTTGTGCA
ATAAAAGTAATACGTAAAATCACACTTATAGGTGTTAATTTGGCTTCTAAACTTATTA
ATTTATAAGATCTTATAACTACATATATTTTATAAAATAATTTAATTATATTTTTAGTCT
CATAATTTTGATCATTCAGCATTTTTAGCTAGTAACTAATCATTTCGGCGGTTTCTGT
TTTCGTGTAACCAGGAGAGATTTGATCGGATTTTTCTCGTGTTTACTAAATTTGTTT
TCAGGTGGGCGGAAAGTGGTATGTGCTACTCAGGTGACTGTTTATAATTAGGGCAA
ATCATCATATTGGCTAATTAATGCCATTTTGATGCCAGAAAGAAAAATTAGCCAATA
GGATGATTCGCCCTAGTTTTCAACAGACACATGAGTGGCAGATACATTTTTCGACC
ATCTCGAAAGAAATCAGGCAAACAGGGGAAAATTGATCAAATTTGTTCTGAATTTG
TTAGAGAAAATATTACATGACAAAATTACAATCTTAACAAGTTAGAGGATTAAATTT
TCAAATGAACATAATTACGGGAGCAAAAATACAATTAAGCCTATAAGATATATATGT
ATATATATAAGATGTTTCTTAAGCTAAGTTGCTAGATTTTATTCTAAAAAGAAGGCTA
TTTAAGTATTTGAATAAAATAAAATTACGAAACTAATTATAATACGTTATTAATAATG
ATAAATTTGTGACTAATTTTTTAAAAAAAATAATATATTTTTAAACTAAGTTGAAAAT
TTTTACTTTTTTCTGAAGAAGCTTATAATTATAAGCTCCAAATCTTAGTTTCCGGATC
GTATAATCTCATTTTCCACAAAATTTACCAAACACTCTGCAACATCTGAAAATTAAG
TTGTATATTACTTTGCCCTATTACAACTACCATTAATTAACGTTTTATGTTCTGATAAT
TAACATATATATATATATACACACACACACATATGTGTGTGTGTTTTTCATAATGCGAT
TTGCAGGTGTGGAAGCATTCAATATGGAGTAAACTTGAAGAGCACATGAACTGCT
GTCGTCGGGGTATTCTCGAGTTCTTCAATATTGTGGGATTCTGACTTGACCCATTGA
GACAAAGTGTGTAGTAGAAACTTCAGTGTATTGGGCTTAATGTTGTAAGAACAGAA
GGGCCCAAAGCCGCCGGAAAAGAGAAGATTGTGTCAGTAGAAAATTACGATCAAG
TGTGTGAAATGAAACCTGTACGCTGT.

The PCR amplification with two primer pairs for sesame materials with seed shattering trait are as follows (5581 bp, including gene upstream, UTR, CDS, and intron sequences):

TGACGGTCCGATTTGTAAGGTGATAGGGATGATTGAAAGATTGTTTCCCCAACCT
TGGATGCATTAGACGGTGCTGCTGGAAGTGGCGTGCTACGACCATAGTAACGTGAT
ACAGGAGGCGCACCACTAGACATAATTAATACAAATTATAATAAACATTAGTTTTTA
TAATTGCCGAAAAAATTAACCTAATTCAATAATTTATGTTGAATTTATGATTATTGTA
CTCATTAATTACTTAAAAATATATAGTTCCTTTGTACAGATATACAAACGGGTCAAGC
ATTGCTTTTAACTTCTTTAGCATATCACATAAATTTTTTTGTTATACTACTACTCCAGT
TTGTCCACTCTTTTTCTGTCAACATTGTCTTCTCTTTACAAATAAGAATGGATAAAC
TTGACTAATTAATTCTACTGAGAAAAAATCACTTGATGAGCTAAAAAAGCTAAAGC
AATGCTTGATCCATTTGTACATGGGCACAAAGAAACTATACATTTTTAATAACTCTTA
TTGAAAATATTATTATTGGAACTATCAGCCTAATATTGTGATTATTGACATAATTATGT
AAATAGTATTATGTGAATACTAGTCTTGTGATTGTTAATATTATTATGTAAATAATGTT
ATTATTGTGAATATTAGTCTAACTTTGCGATTGTTGACATTATTATGTAAATAATATTA
TTATTTAGAATATTGAATAATGTTGTGATTATTGATATTATTATGTAAATAATATTATTC
TTGAAAATATTAGCCTAAATTTTGTGATTATGACATTATTTTGTAAATAATATTATTAT
CGGGAATATTGATCTAATTTTATGATTATTGACATTATTAAATAAATAATATTATCATTT
GAAATATTAGCTTAAAGGTTGTGAATACTGACATTGTTATGTAAATAATGTTATTATT
GAAAATTGTAATCGAATTTTATGATTGTTGACATTATTATGTAAATAATAATATTAATG
AAAAAATACATGAATTACACCACTATGTAAATTACACAATATGATACAATACACACA
AAAATTGGGGGTCATGATGTCATATACCAAATTTCATTCCTAATGTGTTCCAATACTC
GTTTCAATGTGAAACGGTCGAGTTATCCGTGTCAAATTCTATTTTTTCTTTTTTTGTA
GTGTATGACTATTACAGCTCTGAGCATGTCTCAAATAATAATTATCTTTGTTTTTGAA
CAAAAATATTAATTATCTTTAATGCATAGAGAAATGGCTATTTTTAAGCTGTAAAGA
AGCAATTATATGAGAGTGAGAGCTCTTAGTTTAGACAATTATTGATAACTCATCAGC
AAGTGCATTTGATTGTGAGGTCATTATCACCATTAATAAAAGTGATCATAACATCAA
TATCATCAGTAATCATGTTTATGATCAATTAATCAGAATAATTCAATTCGTCTTTGCT
GTGTAATTTGATTTTTTACAATAAAACTTAATTGCATTTTTAGTTCAAAAATTATATA
CAATTATTTGACACTTTAGTCCTGTAATTTATTTGAGTTATAAAAGTAGTCTCATTTT
TTTTAATTCTGCAATTTTAAGACAAAATTGGTCAAATATTCAAAGTTGACCTGAATT
CTGATGCCACTTTTCAGCTAATTTTAAAAATTTCAATCAGATTTGCCATAAACTTGC
AAAATTAAAATAAGGTGAAGAACTAATTTACAACCTAATTAACAAGTTACCAAACT
AATATAAAAATGACTATAATTTTGTTTAAATCTAATAATTATGAAATGAATAATAGCTT
TTCTCATTGAATATTTTATTCATAATATATGATTCTTTTTTTGTGTTCAATAAAGAATAT
GGGGAACCGTACTTGGGATGGAAAGCCTTGTTTGAAAACAGCAAACCCTAGAAAG
GGTTCTAACAATCTGCAATGAAAAAATTCACAGCCCTTTACTTCATCATTTCATAGT
ATATCAAAAGGGGTGGTCCCATCAACATTAATCAATCTCCCTACGTTGTTTCTTTCT
CTTTAAATTCCCTCAAATTTCCTCTCTCTCTCTCTCTCTCTCTCTCTCTATATATATATA
TATATATATATATACATACACTTAAATGCATCAAAGCCCTTAAAAGCTGCAGAGAACC
TAATCAAAGATTGCTTCTGAATTCATCCCTTCAAAGCTAAATCCTCCTCTTTCACCA
GCTCAAGGGTTGTACTTCTTCTCAGAGTACATTTTGATCATGGATTTGAGCCATCAC
AGGTTCACAAACACCACTTGGGAGCCATACAACATTCCCATGGATCAAGGCCATCT
GCTCCAAGATCAAACCCCTTTTGATCATCATCCACAAGCACAATGGCCTTACTTTCC
TCCAATTCACAGCCAAAACGAGCCGTCGGATTCCTCGCCCACGCAACTCCTCCCGT
CTCCGAATTTCGACCAAATGGGCTCTGCAGCCTTCAAAGCGTCAGAAGCCGGAGA
TCATCAAGACGACGAGCCCGAAGAGGAGTTGGGAGCCATGAAAGAGATGATGTTC
AAGATCGCAGCAATGCAACCCGTCGACATCGACCCGGCCACCATCCGCAAGCCCA
GGAGGCGGAACGTCCGGATCAGCGACGACCCGCAGAGCGTCGCGGCCCGCCACC
GCCGTGAGAGGATCAGTGAGAAGATCCGGATCCTCCAGAGACTGGTCCCCGGGGG
CACTAAGATGGACACTGCTTCCATGCTGGATGAAGCCATTCGCTATGTTAAGTTCTT
GAAGAGGCAAATCCGCATACTCCAGGCCAACCACCACCAGCCGCCGTGCATAGGG
ATCGCCACTCCCACCGGCGCCGCCACCGCTGAAGAATGGGTGGCGGTAACAACCA
AGGCCACCGCCACCGCCACCGCGGCAGCAGGGACGTCGTCTTCGTACTTGTTTGG
AGGGAACAACAATAACGATGGGGCAGGTGATGATATATCAATCCCATATGTTTTAAT
TTAACCATAAGTTTTATATATTGGTGATTAGCATTTCACCATATAAATTAGTATATAAA
TTAATTTTGATGATCAAACGTTGTATTATCATAAGGTTTGTATTTAAAAATTCAAAAA
AATCCATGTGATGATAAGAAAAAGGTTTGTTAGGATATTGTTGACTAAGGGGGCAT
CGGAATAATCACAAAACGGTTAGCCTGAGTTATTTATTGTGTGTATATATATAATATAT
GCAAGAAGGGCTTTGGCCCTCATAAACTTCTTTTATTGGCCTATATTTTCCCTGCCC
GAAATAAAACAGTGATGTAAGAAAACTTTGAATATCATGACTATACTCTCTCTAACA
CACACGTAAACCTGCGTGTACGTGTGTGTGTTTGCAGGCTTGTGGAAGAAAGTATA
GCAACCTGCAGAGAGCATATATATATATATGTGTGTGTGTGTGTGTGTTTTATCCCTA
AATATTATGTGGATTGATGATGAATTTGCTGGTGTCTGATGGACCACAAGAGGCCA
AGAATGATTGTGTTCTTTTGCAGTAGCAGACGCCACTTTCACTGCATATACATCATC
ATCATCCATCGATCAAACCCTACTCTTGCAAGAAACTTGGCGAGATTTTCCTCAAC
AACATTAAATAACACCACACACGTTTCTTCTTCTCCCTGCATTCAGACAACAATGTT
CAACCTTATAGCACATTGTCCTGAAATCTGCATCGTACAAATACGACTTCTATGTATA
CATTTTCCAGTGTATATGTGTTGCAATTGTGTGCATACACATACTCAGGGATGTGGC
GTGTAAAAGGAATATATACATATATATATATAATGTCAATGTGTGTAATAAATTGTTAA
GTGTGTAGGATAATTAAATGTATTTTTTCTCCTATAATTTAGGATAAAAATTAAATAC
CAAATACTACCAAAATATAGGATTAGAATACTACCCTAAAAAGTCAAAACAAATATA
ACAAATAACCTAAATTATGAGACCAAAAATATGTTTAACCCTTCATATGTATGACTT
GTATAATTAATGTATTTTACCAGCTAAGTCGTGTTTATTAAACTTGGGATCTTTTCTT
CTCTATATTCCTTTTTTTTTTTTTCAAATTTTCTTCTATATATTCATGTGAGGGGGAAA
TAACACTTTTCGTCCAGTAATTTAGAGCCTTTTCATTTTTGGTTCCTTTAGTTACAAA
ATTCTCATTAGTGGTTCTGTAACTCCTAAAAAATAGCATTTTTACTCCTATAACACAC
TTTTGAGCTGCTAAATTGGTATCCAATGCCTGCATGCATAGTATGATGAATTGTTTGT
TAGATACTGAGTGGGAGCACAGCTTTATATGTCTAATTGTATCTTTACTCAATATTTG
TGCAATAAAAGTAATACGTAAAATCACACTTATAGGTGTTAATTTGGCTTCTAAACT
TATTAATTTATAAGATCTTATAACTACATATATAAAATAATTTAATTATATTTTTAGTCT
CATAATTTTGATCATTCAGCATTTTTAGCTAGTAACTAATCATTTCGGCGTTTTCTGT
TTTCGTGTAACCAAGAGAGATTTGATCGGATTTTTCTCGTGTTTACTAAATTTGTTT
TCAGGTGGGCGAAAAGTGGTATGTGCTACTCAGGTGACTGTTTATAATTAGGGCAA
ATCATCATATTGGCTAATTAATGCCATTTTGATGCCAGAAAGAAAATTAGCCAATAG
GATGATTCGTCCTAGTTTTCAACAGACACATGAGTGGCAGATACATTTTTCGACCAT
CTCGAAAGAAATCAGGCAAACAGGAAAAAATTGATCAAATTTGTTCTGAATTTGTT
AGAGAAAAAATTACATGATCAAATTACAATCTTAACAAGTTAGAGGATTAAATTTTC
AAATGGACAAAATTACGGGAGTAAAAATACAATTAAGCCTATAAGATATATATGTAT
ATATATAAGATGTTTCTTAAGCTAAGTTGCTAGATTTTATTCTAAAAAGAAGGCTATT
TAAGTATTTGAATAAAATAAAATTACGAAACTAATTATAATACGTTATTAATAATGAT
AAATTTGTGACTAATTTTAAAAAAAATTAATATATTTTTAAACTAAGTTGAAAATTTT
TACTTTTTTCTGAAGAAGCTTATAATTATAAGCTCCAAATCTTAGTTTCCGGATCGTA
TAATCTCATTTTCCACAAAATTTACCAAACACTCTGCAACATCTGAAAATTAAGTTG
TATATTACTTTGCCCTATTACAACTACCATTAATTAACGTTTTATGTTCTGATAATTAA
CATATATATATATACACACACACACACATATGTATGTGTGTTTTTCATAATGCGATTTG
CAGGTGTGGAAGCATTCAATATGGAGTAAACTTGAAGAGCACATGAACTGCTGTC
GTCGGGGTATTCTCGAGTTCTTCAATATTGTGGGATTCTGACTTGACCCATTGAGAC
AAAGTGTGTGGTAGAAACTTCAGTGTATTGGGCTTAATGTTGTAAGAACAGAAGG
GCCCAAAGCCGCCGGAAAAGAGAAGATTGTGTCAGTAGAAAATTACGATCAAGTG
TGTGAAATGAAACCTGTACGCTGT.

A primer pair for PCR to identify the sesame shattering resistance trait gene Sihec3 and its allele SiHEC3 is named SSR1. Considering the target genetic marker sites belong to SV (Structure Variation) mutation, this primer pair are designed as follows:

SSR1-F sequence: 5′-TACATGGGCACAAAGAAAC-3′,
SSR1-R sequence: 5′-CTGGTGAAAGAGGAGGATT-3′;

the PCR amplification products for homozygous sesame shattering resistance or shattering type genomic DNA as template are 1724 bp or 684 bp.

In sesame molecular breeding, the primer pair SSR1 is utilized to detect and determine whether a sesame material exhibit the shattering resistance phenotype or not.

A method for distinguishing the sesame shattering resistance gene Sihec3 and its allele SiHEC3 with the primer pair is composed of the following steps:
(1) extracting genomic DNA from a sesame sample;
(2) With the genomic DNA extracted in step (1) as a template, performing a PCR amplification with the primer pair SSR1 and conducting an agarose gel electrophoresis;
(3) determining the phenotype of sesame sample belongs to shattering or shattering resistance based on electrophoretic band type. The criterion is as follows:
if the amplicon is 1724 bp, the tested sample contains SiHEC3, the gene promoter and 5′ UTR sequences are complete, and should be homozygous with shattering phenotype;
if amplicon is 684 bp, the tested sample contains Sihec3, the gene promoter and 5′ UTR sequences are incomplete, and should be homozygous with shattering resistance phenotype;
if there are two amplicons with 1724 bp and 684 bp in length, the tested sample should be heterozygous with shattering phenotype.

In order to accelerate breeding new sesame varieties suitable for mechanized production, Henna Sesame Research Center, Henan Academy of Agricultural Sciences systematically carried out sesame physical and chemical mutagenesis, interspecific hybridization, T-DNA transformation and other germplasm creation technology research since 2008. A seed shattering resistance mutant M7 was preliminary obtained and purified from the constructed sesame mutant library.

In order to further determine the key gene regulating sesame shattering trait, the applicant performed the genetic analysis of seed shattering trait using the mutant M7 and the F₂ and F_{2:3} population derived from the crosses “M7 (shattering resistance)×Yuzhi Dw609 (shattering)” and “M7 (shattering resistance)×Xiangcheng Dazibai (shattering)”. Results have shown that the seed shattering resistance trait in tested population is recessive and controlled by a single gene. Based on self-constructed sesame genome fine map and high-efficient gene mapping techniques, the applicant cloned the target gene Sihec3 and its allele SiHEC3 which regulate the sesame shattering trait and developed the genetic marker for sesame shattering resistance trait.

Overall, the primary techniques and advantages of the present application are as follows: (1) the gene Sihec3 and allele gene controlling sesame seed shattering resistance have been cloned for the first time. Research results indicate that a 1049 bp SV mutation in the promoter and 5′ UTR region of this gene causes the change of seed shattering resistance trait. Cloning of this gene supplies the key information for further exploring the sesame seed development and regulation mechanism; (2) the development of the shattering resistance gene marker SSR1 establishes the basis for identifying shattering resistance trait in sesame germplasm resources and provides the crucial technological support for rapid and precise determination of the seed shattering phenotype and seed purity, as well as for new sesame variety breeding with seed shattering resistance; (3) the shattering resistance gene and its gene marker provided in the application offer vital technical guidance for accelerating molecular-assisted breeding process and breeding new varieties and enhancing mechanization level in sesame production.

In conclusion, the present application provides the significant theoretical foundation for research in seed shattering resistance regulation and seed development mechanism in sesame and other crops. This achievement also supplies valuable genetic resources for sesame molecular-assisted breeding technology system construction and more new shattering resistance varieties breeding. The application has the important scientific and economic applied potential.

### BRIEF DESCRIPTION OF DRAWINGS

FIG. 1 shows a comparison of capsules and seeds between the mutant with seed shattering resistance trait and a sesame material with seed shattering trait;
a: capsules and seeds of the sesame mutant M7 with seed shattering resistance trait; b: capsules and seeds of the sesame germplasm (Yuzhi 11) with seed shattering trait.

FIG. 2 shows a genome-wide association analysis result of the shattering resistance trait in the hybrid population “M7×Xiangcheng Dazibai” according to the present application.

FIG. 3 shows an SV correlation analysis result of the shattering trait in the hybrid population “M7×Xiangcheng Dazibai”.

FIG. 4 shows a comparation between a shattering resistance trait gene Sihec3, and its allele gene SiHEC3, as well as their promoter sequences according to the present application.

FIG. 5 shows an amplification result of SSR1 marker of partial plantlets in the test F₂ population of “Yuzhi NS610×Anhui Qingyangbai” according to the present application;
M: DL2000 (bands from top to bottom are 2000 bp, 1500 bp, 1000 bp, 750 bp, 500 bp, 250 bp, 100 bp in sequence, respectively); lane 1: shattering resistance parents; 2: shattering parents; lane 3-96: some plantlets of test F₂ population. 43 plants are heterozygous with shattering trait (containing bands of 1724 bp and 684 bp); 28 plants are homozygous with shattering trait (containing a band of 1724 bp); 22 plants are homozygous with shattering resistance trait (containing a band of 684 bp).

FIG. 6 shows the amplification result of SSR1 marker with 96 sesame germplasm accessions according to the present application;
M: DL2000 (bands from top to bottom are 2000 bp, 1500 bp, 1000 bp, 750 bp, 500 bp, 250 bp, 100 bp in sequence, respectively); lane 1: shattering resistance parents; 2: shattering parents; lane 3-96: 96 sesame shattering germplasm accessions (containing a band of 1724 bp).

### DESCRIPTION OF EMBODIMENTS

Below is a detailed explanation of the present application, supplemented by the Examples. Before introducing the specific Examples, a brief introduction and explanation of some experimental backgrounds in the following Examples are provided below.

### Sesame Materials

Yuzhi 11, Xiangcheng Dazibai, Anhui Qingyang Bai and other sesame varieties are the main varieties cultivated in sesame production. All these varieties belong to seed shattering materials. M7 is a sesame mutant with seed shattering resistance which was created by Henan Sesame Research Center, Henan Academy of Agricultural Sciences. This line has been effectively utilized for breeding the new sesame variety Yuzhi NS610 which presents the desirable seed shattering resistance trait. All the mentioned germplasm materials are publicly accessible and can be obtained from relevant germplasm resource banks; as a professional research organization, the applicant reserve the relevant sesame germplasm materials for long time.

### Example 1

In order to accurately determine the target gene regulating the shattering resistance trait in sesame and to establish the basis for new variety breeding, a further detailed genetic analysis research on this trait has been conducted. Here is a concise overview of the research process.

### (I) Genetic Background Analysis of Sesame Seed Shattering Trait

During the research, shattering resistance germplasm M7 (shattering resistance) and Xiangcheng Dazibai (shattering) were chosen to construct a hybrid cross (the details of which are presented in Table 1, with the phenotypic comparison illustrated in FIG. 1). The F₁ and F₂ progeny were evaluated for their phenotypes and statistics analyses.

TABLE 1
Trait analysis of hybrid population from the cross between
shattering resistance mutant × shattering genotype material

| | | Shattering | Shattering | |
|---|---|---|---|---|
| | | (plant):shat- | (plant):shat- | |
| | Total | tering | tering | |
| | number | resistance | resistance | |
| Materials | (plant) | (plant) | (plant) | χ² |
| Xiangcheng Dazibai | 15 | 15:0 | — | — |
| (shattering) (P₁) | | | | |
| M7 (shattering | 8 | 0:8 | — | — |
| resistance) (P₂) | | | | |
| F₁ of reciprocal | 18 | 18:0 | — | — |
| cross | | | | |
| F₁ of cross | 18 | 18:0 | — | — |
| combination | | | | |
| F₂ of reciprocal | 266 | 209:57 | 3.67:1 | 1.809 |
| cross | | | | |
| F₂ of cross | 260 | 201:59 | 3.41:1 | 0.739 |
| combination | | | | |

Note:
χ²(0.05, 1) = 3.84.

The Chi-square test results presented in Table 1 reveal that the ratio of the shattering-to-shattering resistance in the F₂ generation of crosses and reciprocal crosses between the shattering resistance mutant and shattering genotype material is approximately 3.67:1 and 3.41:1, respectively. The ratios are consistent with the expected value of 3:1 Mendelian ratio (where χ² is less than 3.84). These results suggest that the shattering resistance trait in the hybrid population of mutant M7×Xiangcheng Dazibai accords with the Mendelian inheritance pattern, is a recessive trait, and controlled by a single gene.

### (II) Gene Sequencing

During constructing the aforementioned hybrid population, samples of young leaves were collected from individual F₂ plantlets for subsequent gene sequencing analysis. Additionally, phenotype investigation and statistical analysis of the F_{2:3} progeny were carried out to determine the homozygous or heterozygous genotype of each F₂ plantlet.

According to the phenotype investigation results, 29 F₂ plantlets with homozygous shattering trait and 35 F₂ plantlets with homozygous shattering resistance were randomly chosen from the tested population. DNA was extracted from leaves of each sample using the improved CTAB method proposed by Wei Libin et al. (“Sesame DNA and RNA synchronous extraction method”, Molecular Plant Breeding, 2008), At the same time, DNA of two parents was also extracted individually.

Finally, genome resequencing of the above 64 materials was performed using Illumina sequencing method, with the sequencing coverage of ≥30×.

### (III) Genome Wide Association Analysis of the Hybrid Population With Seed Shattering Trait

Initially, genome re-sequencing data of each sample was aligned and assembled using the BWA (Burrows-Wheeler Aligner) software, based on the genome data of Yuzhi 11 as the reference genome (reported by Zhang et al. in “Ultra-dense SNP genetic map construction and identification of SiDt gene controlling the determinate growth habit in Sesamum indicum L”, Science Reports, 2016 and Zhao et al. in “Identification of sesamum (Sesamum indicum L.) chromosomes using the BAC-FISH system”, Plant Biology, 2018).

Subsequently, reliable SNPs and InDels were detected and filtered using the Genome Analysis Toolkit (GATK).

A total of 1,620,769 variants including SNPs and InDels between the two parents. Combined with the genome resequencing analysis results of the 64 F₂ plantlets, a generalized linear model (GLM) association analysis was conducted using Tassele 5.0 software.

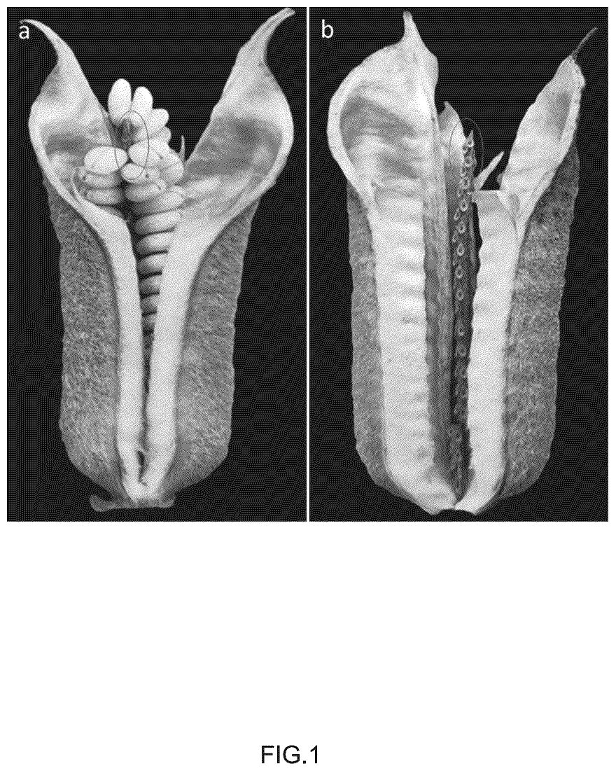

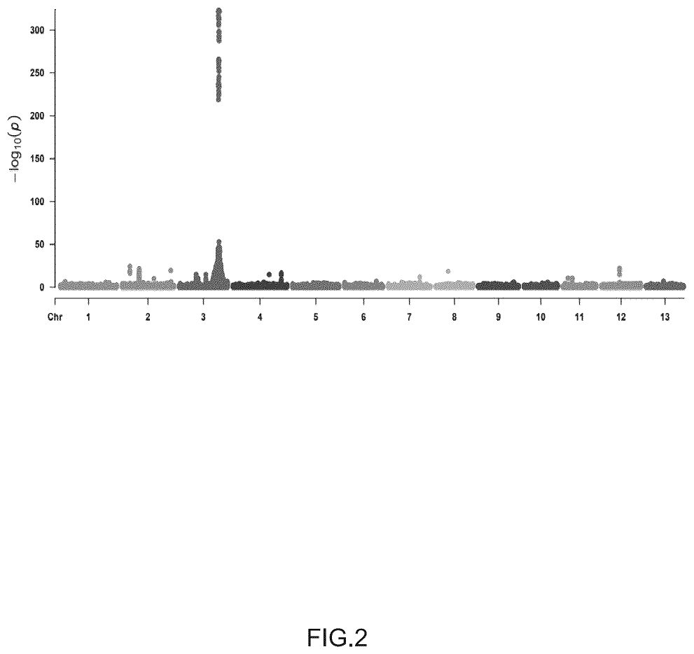

As showed in FIG. 2, the final results revealed that the variants closely associated with the shattering resistance trait (with P value less than E-250) were localized on the 3rd chromosome, ranging from 19,659,488 bp to 20,465,244 bp. This region contains 3,999 SNPs and Indels.

Further, all the candidate SNPs and Indels within this identified region were screened based on the research basis. After filtered, 83 variants remained within the region. Analysis indicates that these 83 variant sites do not cause gene function variation.

### (V) SV Genome Wide Association Analysis of the Hybrid Population With Shattering Resistance Trait

To further determine the target gene, a genome-wide association analysis of SVs in two parents and 64 F₂ plantlets was performed using LUMPY, Manta, and GRIDSS2 software. To enhance the accuracy of SV detection, the results from different software were cross-validated, filtered, and statistically analyzed.

The results indicate that a total of 31,884 SVs, including 2,786 deletions (DEL), 21 insertions (INS), 5729 inversions (INV), 550 duplications (DUP), and 22,798 translocations (TRA) existed in two parents. The length of these SVs ranged from 30 bp to 23 Mb.

Subsequently, a GLM association analysis was conducted with the population phenotype and SV matrix information.

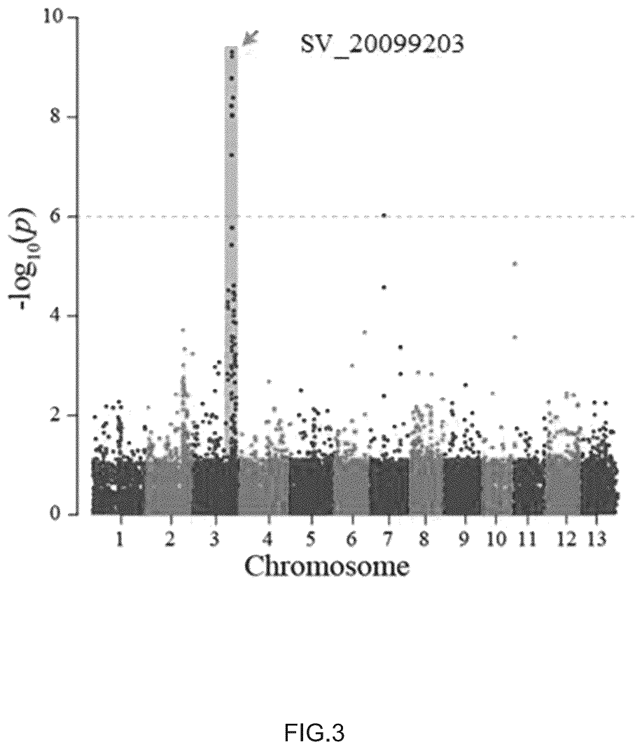

Based on the target region determined by SNP/Indel association and SV matrix information from the hybrid F₂ population, the candidate regions with SV variants were filtered. Elementary results indicated that there are 21 specific SVs within the associated region in the 3rd chromosome of samples with seed shattering resistance trait, as shown in FIG. 3.

Based on the previous work, including the SV matrix library, these 21 SVs were further filtered and screened. The results revealed that the retained 11 SVs were uniquely present in the F₂ samples with shattering resistance trait as detailed in Table 2 below.

TABLE 2
Details of 11 candidate SVs in the shattering resistance F₂ individual plant

| | | | | Explanation | |
|---|---|---|---|---|---|
| | | | | ratio of | |
| | | | Length | variation for | |
| | | SV | of SV | shattering | |
| Chromosome | Location | type | (bp) | resistance trait | Function annotation |
| chrom 3 | 19732809 | INV | 454 | 37.14% | Sindi-0761000-upstream-gene-variant |
| chrom 3 | 19857079 | INV | 44 | 62.86% | / |
| chrom 3 | 19903551 | DEL | 164 | 42.86% | Sindi-0763200-downstream-gene-variant |
| chrom 3 | 19903551 | INV | 160 | 28.57% | / |
| chrom 3 | 19915101 | INV | 46 | 2.86% | / |
| chrom 3 | 19918229 | INV | 36 | 2.86% | / |
| chrom 3 | 19921739 | INV | 147 | 5.71% | / |
| chrom 3 | 19983870 | DEL | 348 | 37.14% | Sindi-0763800-upstream-gene-variant |
| chrom 3 | 20099203 | DEL | 1050 | 88.57% | Sindi-0765000-upstream-gene-variant |
| chrom 3 | 20120488 | DEL | 2313 | 88.57% | Sindi-0765200-downstream-gene-variant |
| chrom 3 | 20132827 | DEL | 50 | 5.71% | Sindi-0765400-downstream-gene-variant |

Note:
“/” locus refers to an intergenic region.

The analysis results in Table 2 indicated that two SVs SV_20120488 and SV_20099203, of the 11 SVs, have the high explanation ratio exceeding 85% for the variation of shattering resistance phenotype. Both SVs belong to DEL type. Of which SV_20120488 is located in the downstream region of Sindi_0765000 gene. SV_20099203 is located in the upstream promoter region of Sindi_0765000 gene. Genome annotation indicates that Sindi_0765000 gene is a homolog of HEC3 gene.

In summary, it is hypothesized that SV_20099203 which involves in a deletion of 1050 bp, impacts the upstream promoter sequence of the Sindi_0765000 gene and possibly causes the alteration of gene promoter's activity, thus changes the gene function, and consequently changes the phenotype from shattering-to-shattering resistance in sesame. Thus, the Sindi_0765000 gene is determined as the target gene associated with the sesame shattering resistance trait and thus designated as Sihec3.

### Example 2

Based on the identification of target gene in Example 1, the gene and upstream promoter sequences of the sesame shattering gene Sihec3 and its allele SiHEC3 were cloned and aligned. A specific process is briefly introduced as follows.
based on the findings from Example 1 and the fine genome map of Yuzhi 11, primer pairs were designed to clone the target gene Sihec3 and allele SiHEC3. Considering the long length of gene and promoter sequence, the full-length gene sequences of Sihec3 and allele SiHEC3 were obtained through a splicing method using different primer pairs. The specific sequences of the primer pairs used in this process are as follows:

Primer 1F forward primer:
5′-TGACGGTCCGATTTGTAAGGTG-3′,
Primer 1R reverse primer:
5′-GAAATTCGGAGACGGGAGG-3′;

according to the preliminary analysis, the amplicons in shattering resistance and shattering germplasm accessions with this primer are 1392 bp and 2432 bp, respectively;

Primer2F forward primer:
5′-AGCTAAATCCTCCTCTTTCACCA-3′,
Primer2R reverse primer:
5′-ACAGCGTACAGGTTTCATT-3′;

according to the preliminary analysis, the amplicons in shattering resistance and shattering germplasms with this primer are 3410 bp and 3408 bp, respectively.

Subsequently, PCR amplification for the genomic DNA of the shattering resistance germplasm M7 and the shattering germplasm Yuzhi 11 was performed using the high-fidelity enzyme PrimeSTAR Max DNA Polymerase (Takara product). The amplification process with a 20 μL PCR reaction system is set as below:
DNA template, 1 μL; dNTP Mix (10 mM each), 0.4 μL; 2×Phanta Max Buffer, 10 μL; Phanta Max Super-Fidelity DNA Polymerase, 0.4 μL;
EvaGreen, 1 μL; Primer F, 0.4 μL; Primer R, 0.4 μL; ddH₂O, 6.4 μL;
a reference PCR amplification reaction program is denaturation at 98° C. for 10 seconds, renaturation at 55° C. for 5 seconds, extension at 72° C. for 3 minutes and 50 seconds, and cycling 30 times; finally, extension at 72° C. for 5 minutes; the amplified product can be directly performed for electrophoresis analysis or stored at 4° C. for further use.

Ultimately, the amplicons were analyzed using electrophoresis. The products were then purified and sequenced (performed by Shanghai Shenggong Biotechnology Co., Ltd.). After sequenced, the sequences of individual amplicons were assembled.

The analysis results revealed that the full-length genomic sequence of the shattering resistance gene Sihec3 is 3406 bp, including two exons and one intron, with the gene sequence present in SEQ ID No. 1. Similarly, the full-length genomic sequence of the allele SiHEC3 associated with the shattering phenotype, is 3441 bp in length and also comprises two exons and one intron.

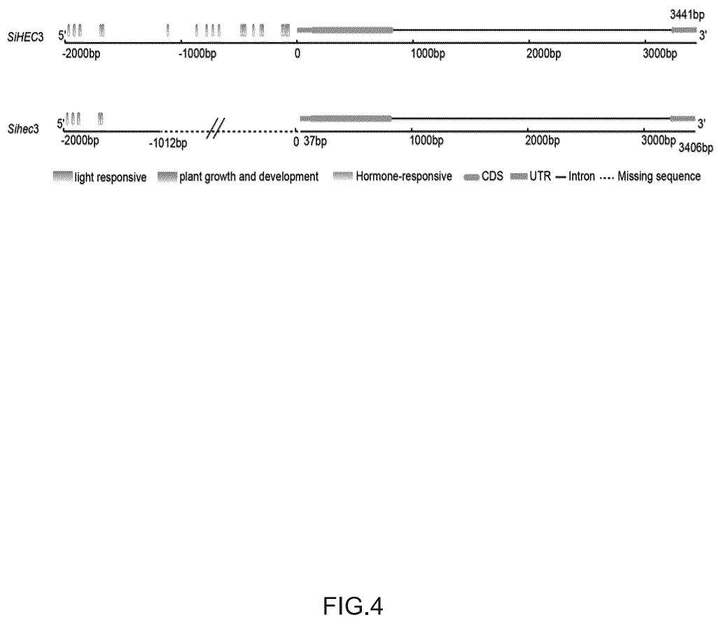

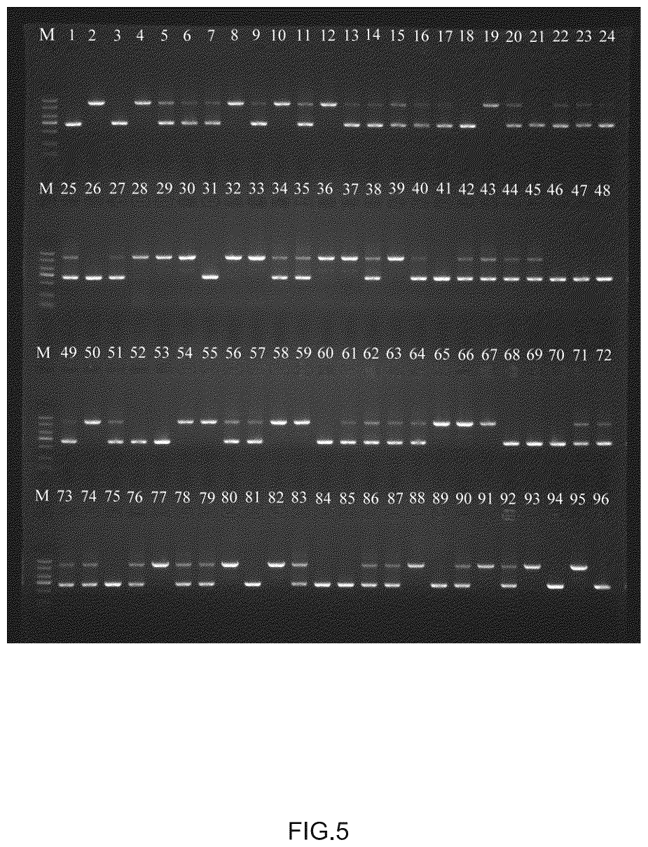

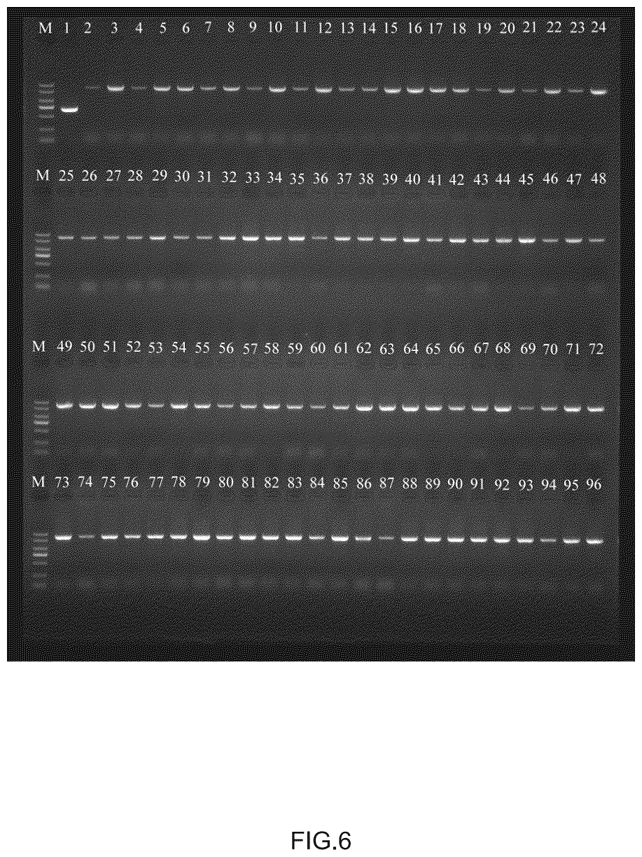

Alignment analysis results shown in FIG. 4 reveals that the upstream promoter region and the 5′ UTR region of the shattering resistance gene Sihec3 are missing 1049 bp, differing from the corresponding sequence of allele SiHEC3. Correspondingly, the phenotype changes from the typical shattering trait to shattering resistance trait. Additionally, there is a single nucleotide polymorphism (SNP), an A to C base mutation at the 111th position within the coding sequence (CDS) of both the Sihec3 gene and SiHEC3 allele. This mutation results in an amino acid change from glutamine to histidine at the 37th position in the protein sequence. Compared with the sequences in other varieties, this mutation site also presents in other germplasm accessions with shattering trait and does not influence the shattering phenotype.

### Example 3

Building on the findings from Example 2, in order to further validate that Sihec3/SiHEC3 regulates the shattering resistance trait, a primer pair for PCR detection was designed, based on sequence differences in the promoter and gene sequences of Sihec3 and allele SiHEC3, in accordance with molecular breeding technology and PCR principles. The primer pair was designated as SSR1 and specifically crafted as follows:

SSR1-F sequence:
5′-TACATGGGCACAAAGAAAC-3′;
SSR1-R sequence:
5′-CTGGTGAAAGAGGAGGATT-3′.

When detecting and determining the phenotype of shattering trait using SSR1 with the primer pair, a specific process is as below:
(1) extracting a genomic DNA from a sesame sample;
(2) taking the genomic DNA extracted in step (1) as a template, performing a PCR amplification using SSR1 and primer pair; during PCR amplification, a 15 μL reference PCR amplification reaction system is as following:
template DNA (50 ng/μL), 1.0 μL; 2×Phanta Max Buffer, 7.5 μL; Phanta Max Super-Fidelity DNA Polymerase. 0.3 μL;
dNTP Mix, 0.3 μL; Forward Primer (10 μM), 0.3 μL; Reverse Primer (10 μM), 0.3 μL; EvaGreen, 0.75 μL;
adding ultrapure water to 15 μL; a reference PCR amplification reaction program is: denaturation at 98° C. for 10 seconds, renaturation at 55° C. for 5 seconds, extension at 72° C. for 120 seconds, and cycling for 30 times; finally, extension at 72° C. for 5 minutes.
(3) Based on the electrophoresis result of PCR product, the sample can be determined to be shattering or a shattering resistant.

It should be noted that the genetic marker site belongs to SV mutation. Therefore, a specific criterion for phenotype determination is that:
when the amplification has a band with a length of 1724 bp, the sample contains SiHEC3 gene with complete promoter sequence and should be homozygous with seed shattering phenotype;
when the amplification has a band with a length of 684 bp, the sample contains Sihec3 gene sequence with the deletion of promoter sequence and should be homozygous with shattering resistance phenotype;
when the amplification simultaneously has two bands with 1724 bp and 684 bp, the sample should be heterozygous with shattering phenotype.

Based on the above method, 96 natural germplasm accessions and 96 hybrid F₂ plantlets of “Yuzhi NS610 (shattering resistance)×Anhui Qingyangbai (shattering)” were randomly chosen for phenotypes detection using the PCR-based genetic marker SSR1.

The results, as partially depicted in FIG. 5), demonstrate that the genotype determined through PCR using the specific primer pair is in accordance with the actual phenotype with 100% accuracy. This result suggests that the gene Sihec3 is indeed the target gene regulating the shattering trait in sesame.

In conclusion, the Sihec3 gene has been identified associated with the regulation of sesame shattering resistance trait and could be used for research in the regulation mechanism of seed growth and development in sesame and other crops. The gene marker SSR1 can be applied in shattering phenotype detection and direct breeding new sesame varieties with enhanced shattering resistance trait.

What is claimed is:

1. A sesame shattering resistance trait regulation gene Sihec3, wherein it is located on the 3rd chromosome of sesame, belongs to a recessive control gene, and has a 100% explanation ratio for a phenotypic variation of shattering resistance trait;
wherein the gene Sihec3 has a length of 3406 bp, contains two exons, and its gDNA is shown in SEQ ID No. 1.

2. A protein encoded by the sesame shattering resistance trait regulatory gene Sihec3 according to claim 1, wherein an amino acid sequence of the protein is shown in SEQ ID No. 2.

3. An allele SiHEC3 of the sesame shattering resistance trait regulation gene Sihec3 according to claim 1, wherein the allele belongs to a complete dominant gene, a phenotype of sesame varieties containing the allele is a normal shattering trait;
wherein the allele SiHEC3 has a length of 3441 bp, contains two exons, and its base sequence is shown in SEQ ID No. 3.

4. An application of the sesame shattering resistance trait regulation gene Sihec3 according to claim 1 in sesame breeding, wherein the gene Sihec3 regulates a phenotype of the sesame shattering resistance trait.

5. A primer pair for PCR detecting and determining the sesame shattering resistance regulation gene Sihec3 according to claim 1 and its allele SiHEC3, wherein the primer pair is named SSR1, and the primer pair is designed as following:
SSR1-F sequence:
5′-TACATGGGCACAAAGAAAC-3′,
SSR1-R sequence:
5′-CTGGTGAAAGAGGAGGATT-3′.

6. An application of the primer pair according to claim 5 in sesame molecular breeding, wherein the primer pair is used for detecting and determining whether the sesame sample is a phenotype of seed shattering resistance trait.

7. A method for detecting and distinguishing a sesame shattering resistance regulation gene Sihec3 and its allele SiHEC3 with the primer pair according to claim 5, comprising the following steps:
(1) extracting a genomic DNA from a to-be-tested sesame sample;
(2) taking the genomic DNA extracted in step (1) as a template, performing a PCR amplification with the primer pair, conducting an agarose gel electrophoresis on an amplification product;
(3) determining whether the tested sesame sample belongs to shattering phenotype or shattering resistance phenotype according to the type of electrophoretic band; and a criterion for determining is:
if an amplified fragment of the sample has a length of 1724 bp, it is determined that the sample is homozygous with shattering phenotype;
if the amplified fragment of the sample has a length of 684 bp, it is determined that the sample is homozygous with shattering resistance phenotype;
if an amplified fragment result simultaneously exists two bands with 1724 bp and 684 bp, it is determined that the sample is heterozygous with shattering phenotype.
